(19) United States
(12) Patent Application Publication
(10) Pub. No.: US 2010/0093018 A1
(43) Pub. Date: Apr. 15, 2010

(54) CELLS EXPRESSING CHIMERIC PROTEINS AND ASSAYS USING SUCH CELLS

(75) Inventors: Xiaohong Hou, Guilford, CT (US); Mingjun Huang, Potomac, MD (US); Wengang Yang, Madison, CT (US)

Correspondence Address: CANTOR COLBURN, LLP, 20 Church Street, 22nd Floor, Hartford, CT, 06103, US

(73) Assignee: ACHILLION PHARMACEUTICALS, INC., New Haven, CT (US)

(21) Appl. No.: 12/597,661

(22) PCT Filed: Apr. 28, 2008
(86) PCT No.: PCT/US08/61779

§ 371 Date: Oct. 26, 2009

Related U.S. Application Data
(60) Provisional application No. 60/914,190, filed on Apr. 26, 2007.
(60) Provisional application No. 60/938,346, filed on May 16, 2007.

(51) Int. Cl. C12Q 1/02; C12N 5/00; C12N 5/071; C12N 15/63; C12N 15/85

(52) U.S. Cl. 435/29; 435/325; 435/370; 435/320.1; 435/455

(57) ABSTRACT

Cells expressing chimeric hepatitis C virus NS4A protein are disclosed, as are vectors and methods for preparing such cells and methods of using the cells and the chimeric proteins therein in screening assays for detecting compounds that alter the associative characteristics of NS4A proteins. Disclosed screening assays are suitable for use in high throughput screening of compound libraries to identify compounds with a exhibiting specific anti-viral activity against hepatitis C virus.

### CROSS REFERENCE TO RELATED APPLICATION

This application claims priority from U.S. provisional application Ser. No. 60/914,190, filed Apr. 26, 2007 and U.S. provisional application Ser. No. 60/938,346, filed May 16, 2007, both of which are hereby incorporated by reference in their entirety.

### FIELD OF THE APPLICATION

This application relates to novel biological tools (including recombinant cells and expression vectors) for identifying compounds with specific anti-viral activity against hepatitis C virus that correlates with their effects on intracellular hepatitis C viral protein-protein interactions, and to assay methods for identifying and characterizing such compounds using such tools.

### BACKGROUND OF THE INVENTION

The hepatitis C virus (HCV) is one of the most prevalent causes of chronic liver disease in the United States. It accounts for about 15 percent of acute viral hepatitis, 60 to 70 percent of chronic hepatitis, and up to 50 percent of cirrhosis, end-stage liver disease, and liver cancer. Almost 4 million Americans (1.8 percent of the U.S. population) have antibodies to HCV (anti-HCV), indicating ongoing or previous infection with the virus. HCV causes an estimated 8,000 to 10,000 deaths annually in the United States. HCV infection occurs throughout the world, and, prior to identification of the virus, represented the major cause of transfusion-associated hepatitis. The seroprevalence of anti-HCV in blood donors from around the world has been shown to vary between 0.02% and 1.23%. HCV is also a common cause of hepatitis in individuals exposed to blood products. There have been an estimated 150,000 new cases of HCV infection each year in the United States alone during the past decade. The acute phase of HCV infection is usually associated with mild symptoms. However, evidence suggests that only 15%-20% of the infected people will clear HCV. Among the group of chronically infected people, 10-20% will progress to life-threatening cirrhosis and another 1-5% will develop hepatocellular carcinoma. Unfortunately, the entire infected population is considered to be at risk for these life-threatening conditions because no one can predict which individual will eventually progress to any of them. An effective vaccine is greatly needed, yet development is unlikely in the near future because: i) lack of efficient cell culture systems and convenient small animal models; ii) a weak neutralizing humoral and protective cellular immune response against HCV; and iii) the marked genetic variability of the virus.

HCV is a small, enveloped, single-stranded positive RNA virus in the Flaviviridae family. The HCV genome comprises approximately 10,000 nucleotides with a single open reading frame (ORF) that encodes an approximately 3000 amino acid polyprotein. The ORF is flanked on the 5′ side by an untranslated region that is a cis-acting RNA element that serves as an internal ribosome entry site (IRES) and at the 3′ side by a highly conserved untranslated sequence necessary for viral replication (3′-NTR). The sequences encoding the structural proteins that form the protein portion of the virus particle and are necessary for viral infection are located near the 5′ end of the ORF. The non-structural proteins, designated NS2 to NS5B, are necessary for intracellular viral replication and are encoded by the remainder of the ORF.

The polyprotein is processed by host cell and viral proteases into three major structural proteins and the non-structural proteins. Several different genotypes of HCV with slightly different genomic sequences have been identified that correlate with differences in response to treatment with interferon alpha.

HCV replicates in the cytoplasm of infected cells, in close association with the endoplasmic reticulum. Upon infection of a cell, incoming positive sense RNA is released and translation is initiated via an internal initiation mechanism. Internal initiation is directed by the IRES; some reports have suggested that full activity of the IRES is seen with the first 700 nucleotides, which spans the 5′ untranslated region (UTR) and the region of the ORF encoding the first 123 amino acids of the polyprotein. All the protein products of HCV are produced by proteolytic cleavage of polyprotein product of the ORF, which is carried out by one of three proteases: the host signal peptidase, the viral self-cleaving metalloproteinase, NS2, or the viral serine protease NS3/4A. The combined action of these enzymes produces three structural proteins (C, E1 and E2) and six non-structural (NS) proteins (NS2, NS3, NS4A, NS4B, NS5A, and NS5B) that are required for replication and packaging of viral genomic RNA. NS3, NS4A, NS4B, NS5A and NS5B are components of the viral replication complex that is involved in viral RNA replication. NS5B is the viral RNA-dependent RNA polymerase that is responsible for the conversion of the input genomic RNA into a negative stranded copy (complimentary RNA, or cRNA); the cRNA then serves as a template for transcription by NS5B of more positive sense genomic/messenger RNA.

The HCV replicon is recombinant construct that, when expressed in a suitable host cell (the product of such expression may also be referred to as HCV replicon), provides an experimentally tractable model of the HCV replication complex that forms in HCV infected cells. The replicon contains, 5′-3′, 1) the HCV-IRES, 2) the neomycin phosphotransferase (neo) gene, 3) the IRES of the encephalomyocarditis virus, which directs translation of 4) a portion of the HCV ORF encoding sequences NS3 to NS5B, and 5) the HCV 3′-NTR. Cells expressing the replicon provide a convenient tool for assessing anti-viral replication effects of compounds, including known anti-viral drugs, and compounds (test agents) being assessed for anti-viral activity. For a more detailed discussion of HCV biology, HCV replicons, and anti-hepatitis-C drug discovery, see Huang M and Deshpande M, “Hepatitis C drug discovery: in vitro and in vivo systems and drugs in the pipeline” Expert Rev Anti-infect Ther, 2(3) 375-388, 2004.

Several institutions and laboratories are attempting to identify and develop drugs to treat HCV infections. Among HCV-specific inhibitors discovered so far, NS5B nucleoside analogs target the polymerase catalytic site of NS5B. These inhibitors block nascent viral RNA synthesis by preventing further elongation after their incorporation into nascent RNA chains. On the other hand, NS5B nonnucleoside inhibitors, which belong to a number of different chemotypes, block the early steps of viral RNA replication by binding to 4 distinctive allosteric sites away from the active site of NS5B. Different from NS5B inhibitors, NS3 protease inhibitors are substrate-based peptidomimetic compounds. They bind to the active site of the enzyme and competitively inhibit NS3 protease activity. Selections of resistance variants with many of these agents using HCV replicon cells have been reported. As expected, the signature resistant mutations of most of inhibitors are located around specific inhibitor-binding pockets and the cross resistance exists among the inhibitors which bind to the same pocket.

The only effective currently marketed therapies for hepatitis C consists of pegylated interferon (IFN), particularly pegylated alpha-interferon, generally in combination with the drug ribavirin. This is suboptimal, especially in patients infected with genotype 1 virus as it reduces the amount of virus in the liver and blood (viral load) in only a small proportion of infected patients. At the present time, the optimal regimen appears to be a 24- or 48-week course of the combination of pegylated alpha interferon and the nucleoside ribavirin, an oral antiviral agent that has activity against a broad range of viruses. By itself, ribavirin has little effect on HCV, but adding it to interferon increases the sustained response rate by two- to three-fold. Nonetheless, response rates to the combination interferon/ribavirin therapy are moderate, in the range 50-60%, although response rates for selected genotypes of HCV (notably genotypes 2 and 3) are typically higher. Among patients who become HCV RNA negative during treatment, a significant proportion relapse when therapy is stopped.

In addition, there are often significant adverse side effects associated with each of these agents. Patients receiving interferon often present with flu-like symptoms. Pegylated interferon has been associated with bone marrow suppressive effects. Importantly, alpha interferon has multiple neuropsychiatric effects. Prolonged therapy can cause marked irritability, anxiety, personality changes, depression, and even suicide or acute psychosis. Interferon therapy has also been associated with relapse in people with a previous history of drug or alcohol abuse.

Side effects of ribavirin treatment include histamine-like side effects (itching and nasal stuffiness) and anemia due to dose related hemolysis of red cells and histamine like side effects.

Direct antiviral agents targeting two viral encoded enzymes, NS3 serine protease and NS5B RNA-dependent RNA polymerase, are currently in development.

The development of resistance to anti-HIV drugs has been a major factor limiting the efficacy of virus-specific therapies in HIV patients. Given the lack of proof-reading mechanism in HCV NS5B polymerase and the high replication rate of HCV in patients, it is well recognized that the emergence of drug resistant HCV variants is inevitable. In fact, the appearance of resistant viruses to anti-HCV drug candidates has already been observed in clinical trials. These resistant HCV viruses may exist as prior variants due to the presence of quasispecies in HCV patients, or may be generated during the treatment period.

Learning from the treatment of HIV patients, combination therapies with agents which act differently and therefore are not cross resistant to each other is believed to be necessary to achieve the sustained suppression of HCV replication. Thus, new drugs with complimentary mechanisms of action to those already available are needed to suppress emergence of viral resistance. Since the testing of large numbers of compounds identified as acting with desirable mechanisms of action is generally required in order to find compounds with maximal efficacy and safety, there is an ongoing need for new, rapid assays for identifying drugs with novel mechanisms of action.

Taken together, the preceding indicates a significant need for methods that will facilitate the identification of new effective small molecule inhibitors of HCV replication, particularly of ones that do not suffer from the above-mentioned drawbacks.

### SUMMARY OF THE DISCLOSURE

It has now been discovered that certain compounds exhibiting anti-viral activity against HCV promote the homo-dimerization of HCV NS4A protein and that the degree of dimerization promoted by each such compound roughly correlates to the anti-viral effects of such compounds in a replicon-based assay for inhibition of intracellular HCV replication, (e.g., the assay of Example 1), which effects are predictive of anti-viral effects against HCV in vivo. This discovery has provided a route to new, convenient, rapid assays that are suitable for use in high throughput formats and are useful for identifying HCV-specific anti-viral compounds that promote NS4A homo-dimerization.

It was first observed that ACH-806, 1-nicotinoyl-3-(4-(pentyloxy)-3-(trifluoromethyl)phenyl)thiourea:

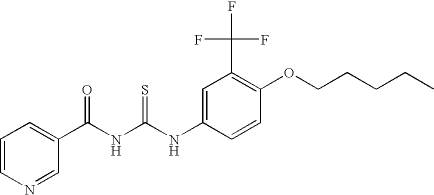

(which has recently been shown to reduce HCV RNA levels in hepatitis C patients) and is also know to reduce replicon replication in vitro (See FIG. 3)) promoted the appearance of an approximately 14 kDa protein band (p14) when anti-NS4A immunoprecipitates of lysates of replicon-containing Huh-7 cell that were separated on denaturing SDS gels were immunoblotted with anti-NS4A antibody (see Example 3 and FIG. 4). Other experiments (see, e.g., Example 8) have demonstrated a lack of cross resistance between ACH-806 and other classes of HCV inhibitors, including nucleoside and nonnucleoside HCV NS5B inhibitors and HCV NS3 protease inhibitors, fueling the desirability of identifying additional HCV-specific anti-viral compounds that promote intracellular formation of p14.

Subsequent experiments have now resulted in the discovery that p14 represents a homo-dimer of the HCV replication protein NS4A. Without wishing to be bound by any particular theory of anti-viral operation, it is believed that by selectively binding to, and promoting the homo-dimerization of, NS4A, ACH-806 prevents the formation of functional HCV replication complexes by interfering with the interactions between NS3 and NS4A in the HCV replication complex. See FIGS. 5 and 6.

This discovery has led to the development of a replicon-free system, adaptable to many cell types, which can serve as the basis of convenient assays that are suitable for implementation in high-throughput formats and that are useful to screen compounds of interest (test agents) for anti-viral, replication inhibitory activity against HCV. Interestingly, while low levels of p14 are detectible in control replicon cells untreated with test agents, p14 is generally not detected in the absence of ACH-806 or another active test agent using the replicon-free cells and methods herein provided.

The cells and methods provided herein are preferably replicon-free cells, which, in general outline, comprise expression vectors directing the intracellular expression of at least one, and in many of the preferred embodiments two (different) chimeric NS4A proteins, each of which proteins comprises an NS4A protein fused to (preferably as a contiguous amino acid sequence) a protein tag that can be used to isolate, immobilize, or otherwise detect the presence of the chimeric NS4A in cells, cell lysates, and fractions thereof. Preferred cells comprising two populations of chimeric NS4A with two distinctly detectible tags, provide a convenient and versatile platform for assaying test agents for the promotion of the formation of NS4A-NS4A dimers. Such dimers can be readily detected by the presence in a single linked molecular assembly (i.e., at least two NS4A and/or chimeric NS4A proteins that are connected together so that they co-isolate, co-attach, or co-localize), of both protein tags, as can be detected by interactions between the two proteins that can only be detected when the proteins are joined together by covalent or non-covalent bonds. Non-limiting examples of such detectible interactions between chimeric tagged NS4A proteins include:

1) Attachment to a substrate via an attachment functionality (e.g., an affinity tag, or an epitope tag immunoreactive with an antibody pre-attached to the substrate) of one of a dimeric pair of differently-tagged chimeric NS4A proteins, followed by detection on the (preferably) subsequently washed (to remove unattached proteins) substrate of another of the pair of attached differently-tagged chimeric NS4A proteins, this one lacking the particular attachment functionality, but comprising a distinct detectible functionality, such as a radioactive functionality, a fluorescent functionality, or an immunodetectible functionality such as a distinct epitope tag;

2) An interaction between protein tags each comprising one of two protein fragments that, when the fragments are joined together in a single linked molecular assembly, can interact in a complementary fashion to provide a detectible function, such as fluorescence or an enzymatic activity (e.g., proteolytic or lactamase activity); and

3) A proximity interaction between two fluorescent tags, such as detectible fluorescence resonance energy transfer (FRET) resulting in a different ratio of fluorescent energy emissions produced by the two fluorescent tags (e.g., CFP and YFP illuminated by single-laser excitation at 458 nm) when they are dimerized as when both are present but are not closely associated (linked) with each other.

Therefore, in various aspects there is here provided:

A cell that intracellularly co-expresses

a first chimeric tagged HCV NS4A protein comprising a first contiguous amino acid sequence consisting essentially of HCV NS4A protein and a first protein tag; and

a second chimeric tagged HCV NS4A protein comprising a second contiguous amino acid sequence consisting essentially of HCV NS4A protein and a second protein tag,

wherein the first protein tag and the second protein tag are different.

A cell that intracellularly co-expresses a first chimeric tagged HCV NS4A protein and a second chimeric tagged HCV NS4A protein, such that each of the first chimeric tagged HCV NS4A protein and the second chimeric tagged HCV NS4A protein comprises a contiguous amino acid sequence consisting of HCV NS4A protein and a protein tag, and the protein tag of the first chimeric tagged HCV NS4A protein is a different protein tag than the protein tag of the second chimeric tagged HCV NS4A protein.

In the above cell the first chimeric tagged HCV NS4A protein is expressed at a first concentration within the cell and the second chimeric tagged HCV NS4A protein is expressed at a second concentration within the cell, the first concentration preferably being within about an order of magnitude of the second concentration, the first concentration and the second concentration preferably being about equimolar.

### BRIEF DESCRIPTION OF THE DRAWINGS

FIG. 1. In this figure, the nucleotide sequence encoding NS4A (SEQ ID NO:1) is in the upper 5′-3′ strand and is the only part of the sequence therein set forth in lower case. It begins with agc and ends with tgc. The amino acid sequence which is encoded by SEQ ID NO:1 is SEQ ID NO:2, which appears in standard single letter amino acid format as part of the product of the only open reading frame in the figure, which is the middle of the three reading frames in each group of three therein. The NS4A amino acid sequence (SEQ ID NO:2) begins with the included fifth residue of the middle reading frame (S) and ends with the included fifty-ninth residue (C) of that reading frame.

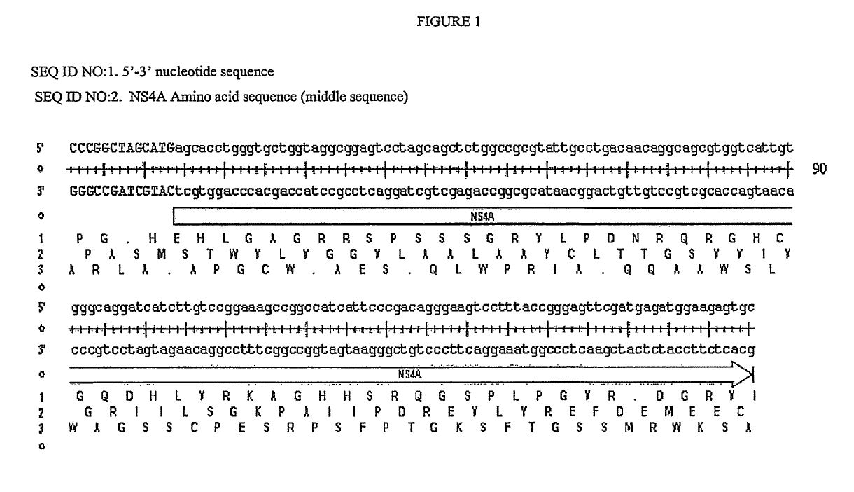

FIG. 2. Diagram showing locations of mutations conferring resistance to compound promoting the homo-dimerization of NS4A (ACH-806), which mutations have been mapped to the N-terminus of HCV protein NS3. Sequencing of ACH-806 resistant clones showed mutation of Cys-16 to Ser-16 and Ala-39 to Val-39. NS3 amino acid residues C16 and A39 are indicated in the drawing. NS3 protease domain (pdb code: 1NS3) is colored gray where beta-strands (cyan) and α-helices (red) are shown as ribbons; catalytic site residues are colored red (Asp 81), black (His 57) and green (Ser 139); structural zinc ion is displayed as a red sphere; NS4A is colored magenta; and, the CPK representation of resistant mutants (Ala 39 and Cys 16) is in blue. The distance between Cys 16 (Cb) of NS3 and Val 26 (CG1) of NS4A and Ala 39 (Cb) of NS3 and Gly 21 (Cα) of NS4A is 4.1A and 7.3A, respectively.

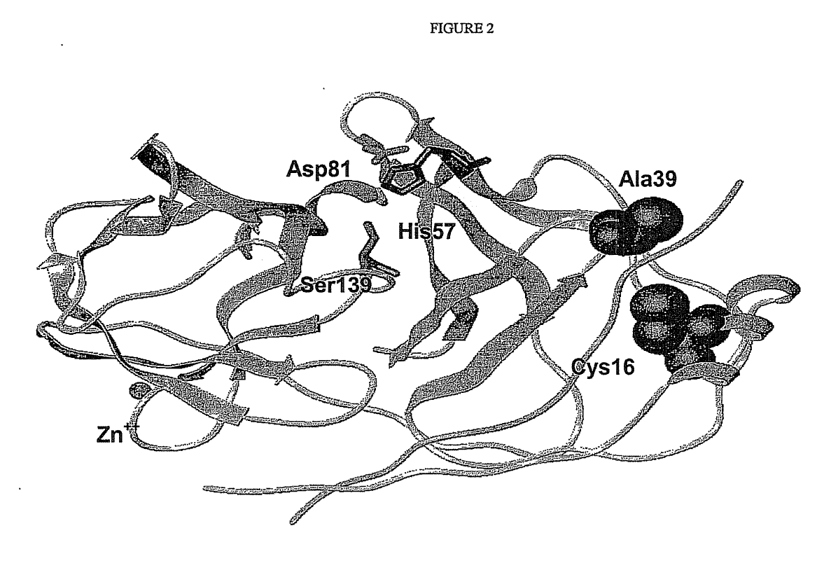

FIG. 3. ACH-806 is a potent inhibitor of functional replicase complex production. 16 hour treatment of replicon cells with ACH-805 resulted in more than 50% reduction of activity of replicase activity at concentrations as low as 50 μM.

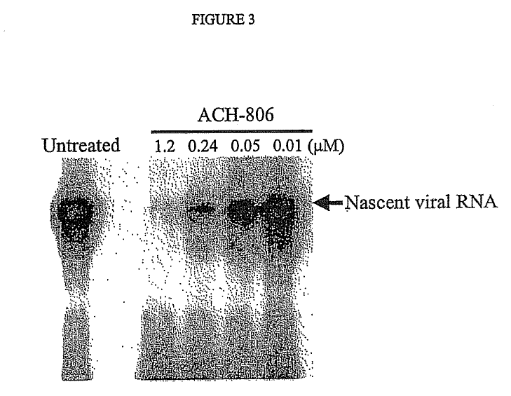

FIG. 4. The specific alterations of viral protein composition and viral-viral protein interaction in ACH-806 treated replication complexes. Replication complexes were isolated from replicon cells (Huh 9-13) after exposure to ACH-806 (0.2, 4 and 10 micromolar), NS3 serine protease inhibitor (20 micromolar), or NS5B RdRP nucleoside inhibitor (20 micromolar) overnight and were subjected to SDS/PAGE after solubilizin with the sample buffer (Laemmli sample buffer supplemented with 5% beta-mercaptoethanol). Following transfer of the proteins to the membrane, the membrane was probed sequentially with the antibody labeled on the left, after stripping off the previous antibody. Molecular weight markers are indicated on the right.

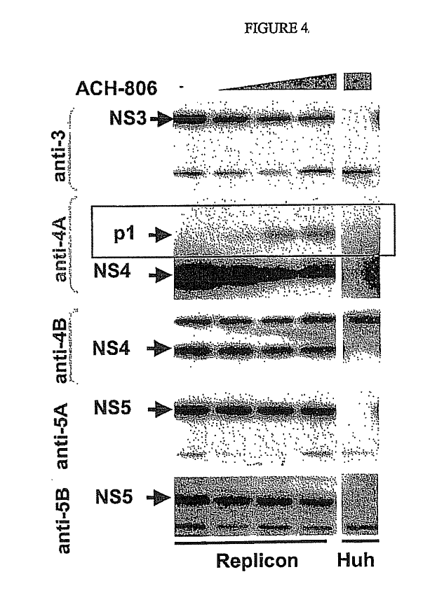

FIG. 5. Western blot of HCV replicon cells treated for 16 hours with ACH-806. Cell lysates from Huh-9-13 cells after exposure to ACH-806 (2 μM), NS3 serine protease inhibitor (20 μM), NS5B RdRP nucleoside inhibitor (20 μM), and nucleoside inhibitor for 8 hours were immunoprecipitated with and The SDS-PAGE gel was immunoblotted with anti-5B, anti-5A, anti-4A, and anti-3a antibodies. A does-dependent accumulation of a new NS4A containing species (p14) is observed. A dose-dependent reduction in mature NS3 and NS4A replicon proteins is also observed. There is no significant reduction in NS4B, NS5A or NS5B levels.

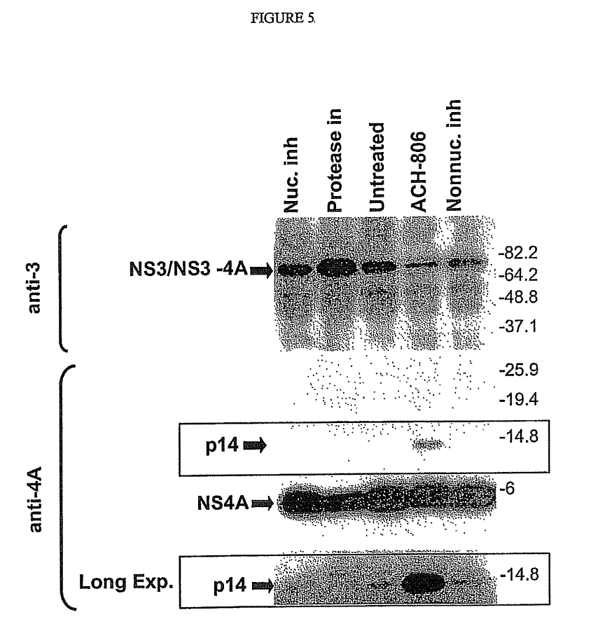

FIG. 6. Cell lysates from Huh-9-13 cells after exposure to ACH-806 (0.2, 1 and 5 μM) overnight were subjected to immunoprecipitation with anti-NS4A antibody. Immunoprecipitates were solubolized and resolved on SDS/PAGE. Following transfer of the proteins to the membrane, the membrane was probed sequentially with the antibody as indicated on the right after stripping off the previous antibody.

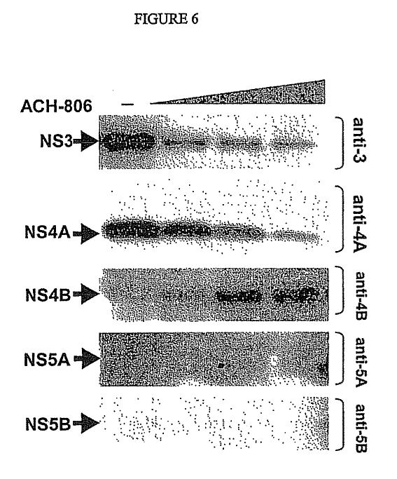

FIG. 7. Cell lysates from Huh-9-13 cells after exposure to ACH-806 (5 μM) overnight were dived into two parts, each part subjected to immunoprecipitation with anti-NS4A (top) or anti-NS4B antibody, respectively. Immunoprecipitates were solubolized and resolved as above followed by immunoblotting with anti-NS4A antibody.

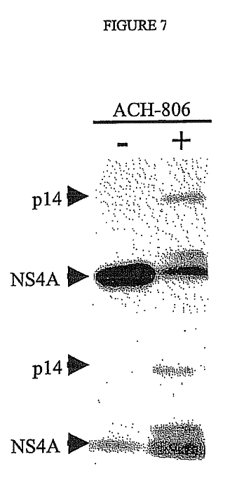

FIG. 8. Cell lysates from Huh-9-13 cells after exposure to ACH-806 (5 μM) overnight were divided into two portions. The smaller portion was diluted with the sample buffer (Input) and the larger portion was subjected to immunoprecipitation with anti-NS4A antibody followed by solubolization with the sample buffer (Pulldown). After resolving on SDS/PAGE, the proteins were immunoblotted with anti-NS3 antibody. The densities of NS3 were obtained by the densitometry and were standardized with that of the untreated input as shown at the bottom of each lane. The ratios of the intensity between the untreated and treated sample were calculated for the input as well as for the pulldown and are shown in the chart. Position of NS3, NS3-4A, p14, NS4A, NS4B, NS5A and NS5B are shown by arrows on the left.

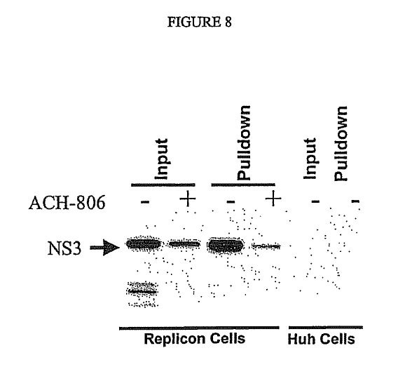

FIG. 9. Western blot of Huh-7 cells transfected with an NS3, NS4A, Flag-NS4B, NS5A, or NS5B expression plasmid. Upon ACH-806 treatment p14 was greatly increased. No significant change was detected in cells transfected with plasmids other than NS4A. Huh-7 cells were transfected with plasmids encoding NS3, NS4A, NS4B with a N-terminal Flag, NS5A and NS5B, respectively, and were lysed in the sample buffer after exposure to ACH-806 (2 μM) overnight. The lysates were resolved on SDS/PAGE and were immunoblotted from the top to bottom with anti-NS3, anti-NS4A, anti-Flag, anti-NS5A and anti-NS5B antibody, respectively.

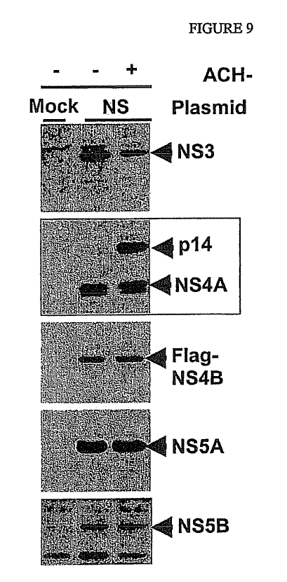

FIG. 10. Effect of strong denaturant on p14. Western blot of Huh-7 cells transfected with NS4A (top) and replicon cells (bottom) p14 became almost undetectable when 8M urea was added to the sample buffer. Cells, either Huh-7 transfected with a plasmid encoding NS4A or Huh-9-13, were treated as above. Transfected Huh-7 cells were lysed with the sample buffer (left) or the sample buffer plus 8 M urea (right). RCs were isolated from Huh-9-13 cells and were solubolized with the sample buffer (left) or the sample buffer plus 8 M urea (right). All samples were resolved on SDS/PAGE followed by immunoblotting with anti-NS4A antibody.

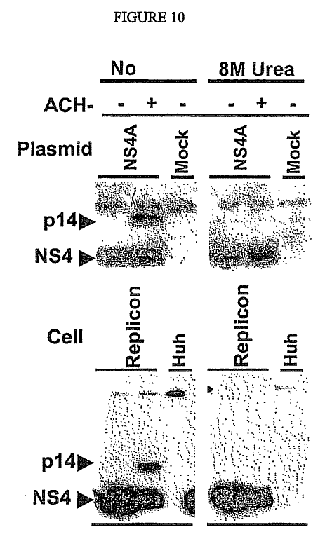

FIG. 11. Western blot of Huh-7 cells transfected with an NS3, NS4A, Flag-NS4B, NS5A, or NS5B expression plasmid. Upon ACH-806 treatment p14 was greatly increased. No significant change was detected in cells transfected with plasmids other than NS4A. Two plasmids, one encoding NS4A and the other encoding NS4A with a C-terminal HA (hemagglutinin) tag (NS4A-HA), were transfected alone or together in 1:1 ratio to Huh-7 cells. Following treatment as above with ACH-806, cells were lysed and the lysates were subjected to immunoprecipitation with anti-NS4A antibody. The immunoprecipitates were resolved on SDS/PAGE followed by immunoblotting with anti-NS4A antibody. The carton at the top illustrates the expected species of dimers, NS4A (open box) homo-dimer (species A), NS4A-HA (open box with a shaded box at its C-terminus) homo-dimer (species C), and NS4A and NS4A-HA hetero-dimer (species B), if two can form a dimer Positions of every protein species are shown by arrows on the left or the right.

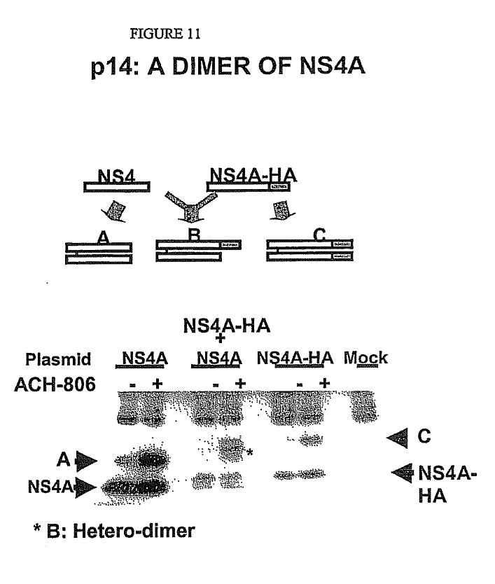

FIG. 12. Selective binding of ³H-ACH-119 to NS4A expressed in HCV replicon cells. Huh-9-13 cells or their parental Huh-7 cells were treated with 40 nM ³H-ACH-119, a compound structurally related to ACH-806 for 20 hours followed by photo lysis, cell lysis, immunoprecipitation using anti-NS3 (top, Lane 1 and 2) or anti-NS4A antibody (bottom, Lane 1 and 2), SDS/PAGE and fluorography. As controls, the cells were treated with 40 nM ACH-119, and were metabolically labeled simultaneously with ³⁵S-met/cystein for 20 hours. Following lysis, a small aliquote were subjected to immunoblot analysis with anti-NS3 (top, lane 5 and 6) or anti-NS4A (bottom, Lane 5 and 6) antibody and the rest were subjected to immunoprecipitation with anti-NS3 (top, lane 3 and 4) or anti-NS4A (bottom, lane 3 and 4) antibody, SDS/PAGE and fluorography. Positions of NS3 and NS4A are indicated on the left and the molecular weight markers are shown on the right.

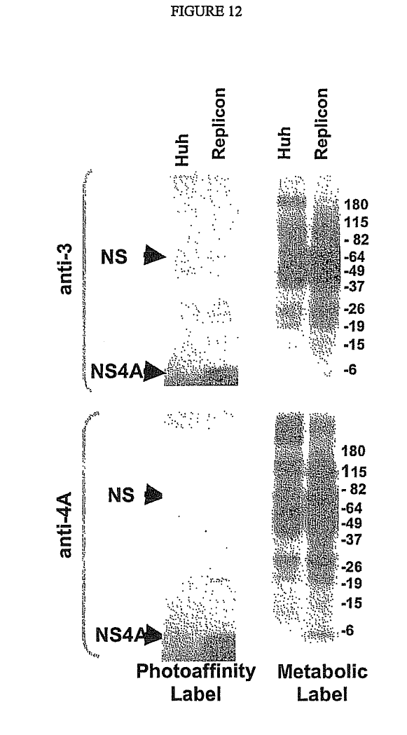

FIG. 13. Approximately 0.25 μM of the synthetic full length NS4A or the recombinant NS3 with its N-terminus fused a central domain (amino acid 21-33) of NS4A (designated as Cofactor domain-NS3) were incubated with 40 nM ³H-ACH-118 at 30° C. for one hour followed by photo lysis, SDS/PAGE, coomassie blue staining (left) and finally fluorography (right). Positions of NS4A and cofactor domain-NS3 are indicated in the middle and the molecular weight markers are shown on the left.

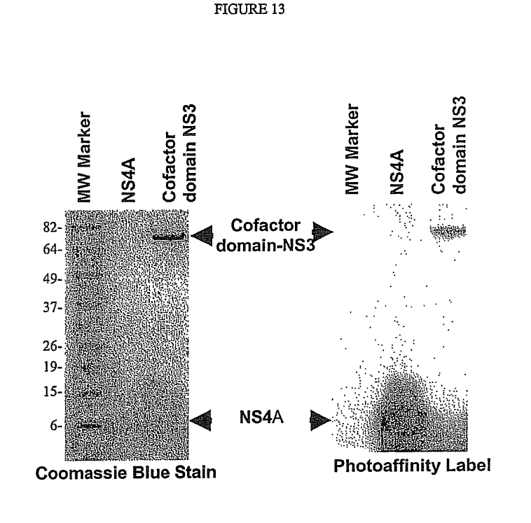

FIG. 14. Approximately 0.66 μM of the synthetic full-length NS4A were incubated with 0.2 μM ³H-ACH-119 in the absence or the presence of the decreasing concentrations (20, 5, 1.25, and 0.3 μM) of the cold compound as indicated at 30° C. for one hour followed by photo lysis, SDS/PAGE and fluorography. Position of NS4A is indicated on the left.

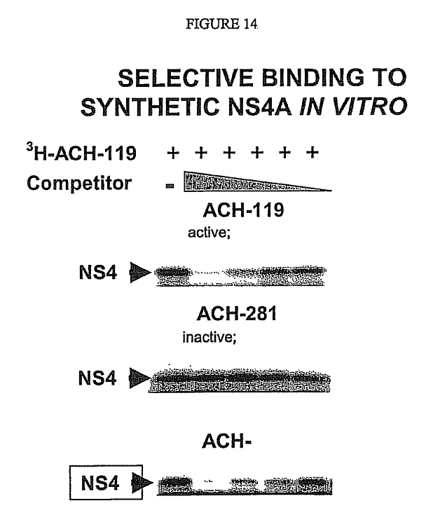

FIG. 15. Chart showing alterations in viral-viral protein interactions. Communoprecipitation was used to examine interactions between viral proteins. The only significant changes noted were changes in the interaction of NS4A with NS3 and NS4B.

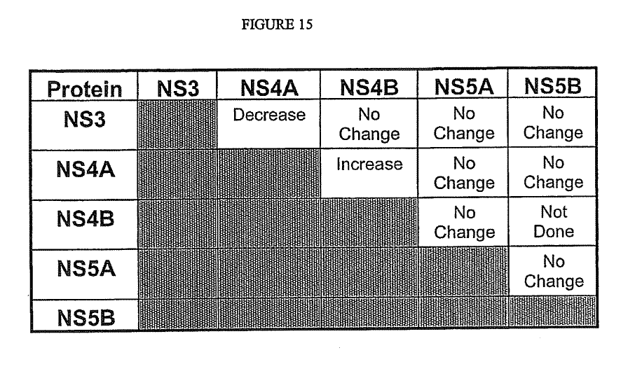

FIG. 16. High-throughput screening assay for NS4A Antagonists. (1) Plates are coated with anti-Tag 1 antibody. (2) Huh-7 cells expressing two different NS4A molecules, each with different Tag labels are lysed to obtain NS4A Tag-labeled protein (4) Test compound is added to the lysate. (4) Lysate is added to the plate. Monomer Tag 1-labeled NS4A and dimer NS4A in which at least one monomer is Tag 1-labeled binds to the plate. (5) Unbound NS4A is washed away. (6) Anti-Tag 2 antibody is added to the plate. The antibody binds to bound dimer in which one monomer is tag-labeled. (7) Unbound antibody is removed prior to assay detection. (8) Plates are developed.

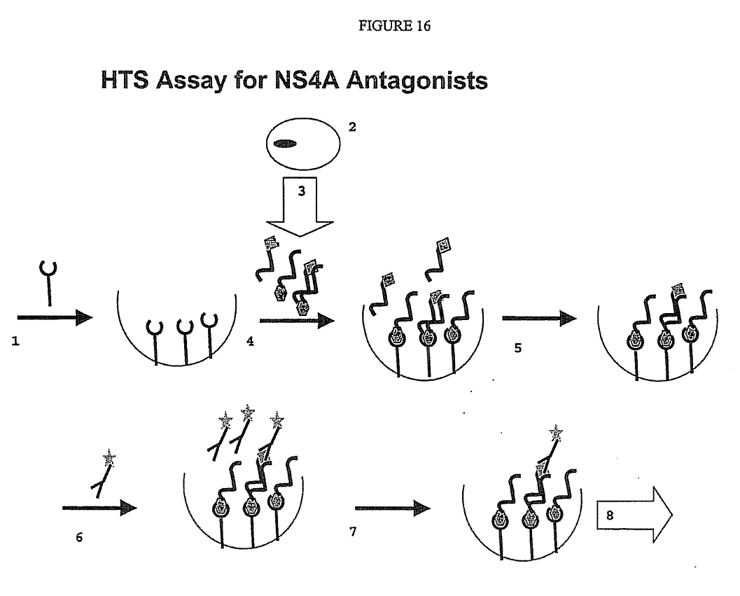

FIG. 17. Construction of NS4A-cFLAG. Clone HCV NS4A (1b) with C-terminal FLAG antibody tag at NheI/EcoRI site of pCl-neo vector (Promega).

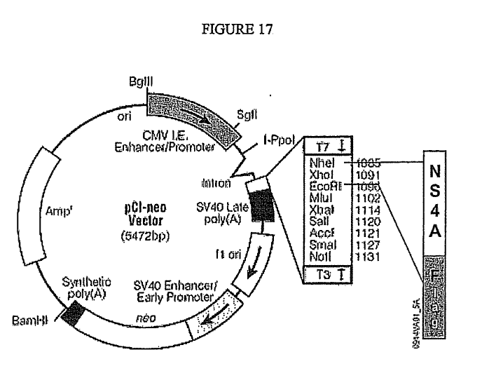

FIG. 18. Construction of NS4A-cV5. Clone HCV NS4A (1b) with C-terminal V5 antibody tag at NheI/EcoRI site of pcDNA3.1 (+) (Invitrogen).

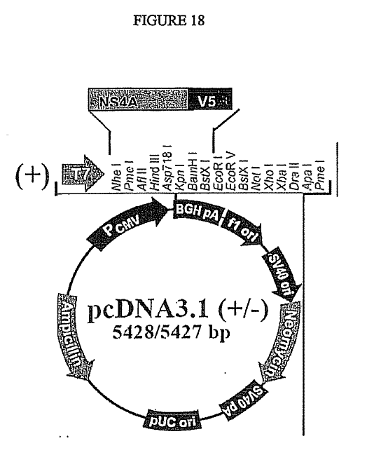

FIG. 19. Western blot of lysate from cell co-expressing NS4A-cV5 and NS4A-FLAG. Upon ACH-806 treatment, dimer p14 is readily detected in the cells transfected with either NS4A-cFlag or NS4A-cV5.

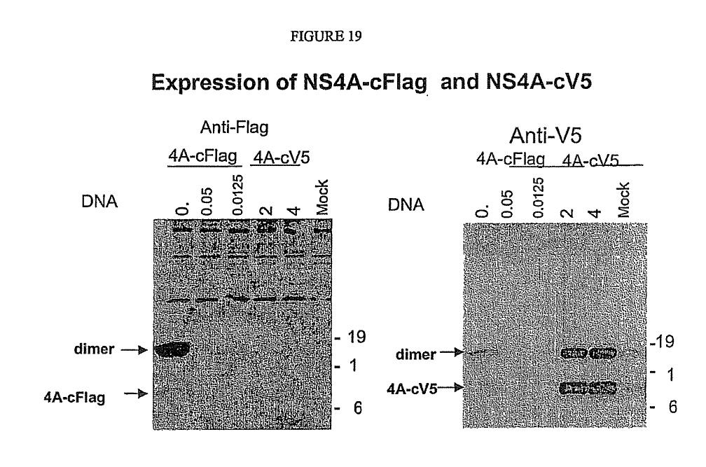

FIG. 20. NS4A Dimer Induction with compound in NS4A-cV5/NS4A-FLAG co-transfected cells. Cells are treated with increasing concentrations of acylthiourea or acylthiourea, EC50's shown. All compounds except 9652, which has an EC50>1000 nM, exhibit concentration dependent p14 dimer formation.

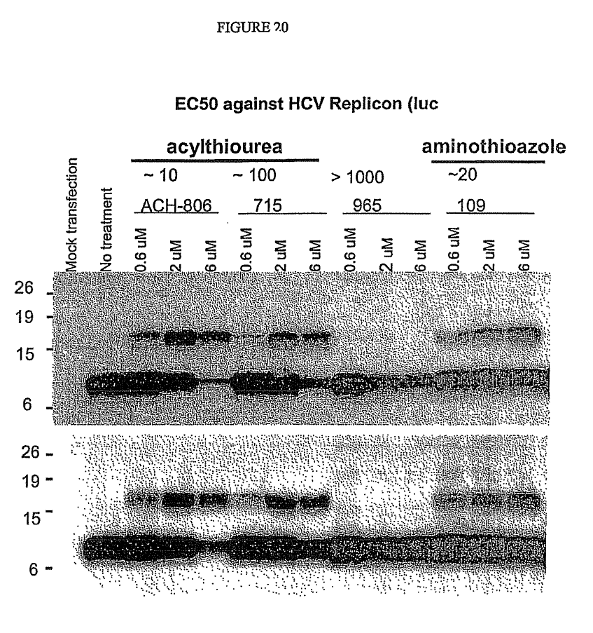

### DETAILED DESCRIPTION OF EMBODIMENTS

Molecular cloning techniques and reagents are so highly developed and commercial kits facilitating such techniques are so commonplace, and the level of ordinary skill typical of skilled molecular biologists is so high that detailed recitation of these techniques and reagents here is unnecessary.

Preferred host cells for various uses and compositions disclosed herein are eukaryotic cells, preferably vertebrate cells, more preferably mammalian cells. Highly preferred cells include human hepatocellular carcinoma Huh-7 (Huh7) cells, African green monkey COS cells and Chinese hamster ovary CHO cells. Preferred COS cells are Cos-1 cells. CHO-K1 lysine auxotrophic Chinese hamster ovary cells are particularly preferred for use as HCV replicon-free cells expressing HCV NS4A protein, preferably chimeric tagged HCV NS4A proteins in accordance with the disclosure herein. Such host cells can be obtained commercially, e.g., from the ATCC, Manassas, Va. Preferably host cells for expression of the chimeric NS4A proteins disclosed herein do not comprise hepatitis C virus replication complexes or replicons. Cells may be transiently or stably transfected with vectors disclosed herein. Cells are preferably prepared as cultures comprising a plurality of cells. The cells expressing such vectors are preferably prepared so that, upon treatment of the culture by incubation for about 20 hours in culture medium comprising about 6 micromolar ACH-806, dimers of chimeric tagged HCV NS4A proteins can be detected in the cells or in lysates thereof. Thus, lysates of the cells of such cultures may be useful in certain of the assays herein provided.

Any of a variety of protein tags are suitable for the chimeric tagged HCV NS4A proteins used in many of the preferred embodiments disclosed herein. See, for example, Terpe K., “Overview of tag protein fusions: from molecular and biochemical fundamentals to commercial systems,” Appl Microbiol Biotechnol. 2003 January; 60(5):523-33; and Brizzard B and Chubet R, “Epitope tagging of recombinant proteins,” Current Protocols in Neuroscience, John Wiley & Sons, Inc., 2001; Chapter 5:Unit 5.8.

Particularly preferred tags include the FLAG tag (Asp Tyr Lys Asp Asp Asp Asp Lys—SEQ ID NO:3—see, e.g., Einhauer A, Jungbauer A, J Biochem Biophys Methods. 2001 Oct. 30; 49(1-3):455-65) and the V5 tag (Gly Lys Pro Ile Pro Asn Pro Leu Leu Gly Leu Asp Ser Thr—SEQ ID NO:4—see, eg., the GATEWAY pcDNA 3.1/nV5 DEST expression vector incorporating a V5 tag, available from INVITROGEN. Other preferred tags include myc, Tab2, and HA epitope tags (see, e.g., Crusius K, et al., “Tab2, a novel recombinant polypeptide tag offering sensitive and specific protein detection and reliable affinity purification” Gene. 2006 Oct. 1; 380(2):111-9). Other preferred tags include chitin binding protein, maltose binding protein, streptavidin-binding peptide, polystyrene-binding peptide, and poly(His) (preferably comprising one or more hexa-histidine motifs) affinity tags. See, e.g., Lamla T and Erdmann V A; “The Nano-tag, a streptavidin-binding peptide for the purification and detection of recombinant proteins”; Protein Expr Purif. 2004 January;33(1):39-47; Kumada Y, et al., “Development of a one-step ELISA method using an affinity peptide tag specific to a hydrophilic polystyrene surface” 2007 J Biotechnol; 127(2):288-99; Kumada Y, et al.; “Protein-protein interaction analysis using an affinity peptide tag and hydrophilic polystyrene plate”; J Biotechnol. 2007 128(2):354-61; and Ljungquist C, et al., “Immobilization and affinity purification of recombinant proteins using histidine peptide fusions.” Eur J Biochem. 1989 186(3):563-9. Preferred fluorescent tags include GFP, YFP, CFP, or OFP (Green, Yellow, Cyan or Orange Fluorescent Protein) fluorescence tags, and luciferase tags, as well as protein tags conjugated to non-protein fluors e.g., as detailed below.

Preferred assays for the detection of NS4A-NS4A dimerized chimeric NS4A proteins are homogeneous assays, as well as Enzyme-Linked ImmunoSorbent Assays (ELISA) and Fluorescence-Linked ImmunoSorbent Assays (FLISA) both of which may be heterogeneous assays. Texts elaborating upon the details of useful assay technologies include the Handbook of Assay Development in Drug Discovery, Minor L K, Editor, CRC; 2006 (see, e.g., Chapter 17, Garippa R J “The emerging role of cell-based assays in drug discovery” pp 221-226 and Chapter 18, Hoffman, A F “The preparation of cells for high content screening” pp 227-242). Also see Protein-Protein Interactions: Methods and Applications, Haian Fu, Editor, Humana Press; 2004 (see, e.g., Chapter 11, Park S-H and Raines R T “Fluorescence Polarization Assay to Quantify Protein-Protein Interactions” pp 161-166 and Chapter 13, Vikis H G and Guan K-L “Glutathione-S-Transferase-Fusion Based Assays for Studying Protein-Protein Interactions” pp 175-86); Protein-Protein Interactions: A Molecular Cloning Manual, Golemis E and Adams P D, Editors, Second Edition, Cold Spring Harbor Laboratory Press 2005 (see, e.g., Chapter 10, Verveer, P J et al., “Imaging Protein Interactions by FRET Microscopy” pp 181-214); and Yoshitake K et al., “Dimerization-based homogeneous fluorosensor proteins for the detection of specific dsDNA.”; Biosens Bioelectron. 2008, 23(8):1266-1271.

See also Handbook of Drug Screening, Seethala R, Editor and Fernandes P, Editor; Informa Healthcare 2001; Chapter 4, Seethala R “Homogeneous Assays for High-Throughput and Ultrahigh-Throughput Screening” pp. 69-128; Homogeneous Assay Development, Promega Notes 74, 3-6, 2000; McCormick M “FRET based homogeneous assay of S•TAG fusion proteins” InNovations 10, 10, 1999; and Whitfield J et al., “High-throughput methods to detect dimerization of Bcl-2 family proteins” Anal Biochem. 2003 322(2):170-178; as well as “ALPHASCREEN Amplified Luminescent Proximity Homogeneous Assay” (at http://las.perkinelmer.com/applicationssummary/applications/Principles+of+AlphaScreen.htm as of the international filing date hereof). The Perkin Elmer ALPHASCREEN homogeneous assay provides a preferred detection system that comprises microscopic donor and acceptor beads that are each coated with a layer of hydrogel providing functional groups for bioconjugation. A representative homogeneous assay using this technology can be carried out with cells expressing both chimeric HCV NS4A tagged with streptavidin-binding peptide and chimeric HCV NS4A tagged with V5 epitope tag and with donor beads conjugated to streptavidin (these are available pre-conjugated from the manufacturer) and acceptor beads conjugated to anti-FLAG antibodies (readily prepared by incubating commercially available IgG anti-FLAG antibodies with acceptor beads coated with protein A, which are available pre-conjugated from the manufacturer). When NS4A/V5-NS4A-FLAG dimers of the V5- and streptavidin-binding peptide-tagged chimeric HCV NS4A proteins are present, the presence of both tags in a linked molecular assembly and the affinity between the two different dimerized tags and the two different bead coatings bring the donor and acceptor beads into close proximity. A cascade of chemical reactions is thus initiated that produce a greatly amplified light signal upon laser excitation at 680 nm. In outline, a photosensitizer in the donor bead converts ambient oxygen to a more excited singlet state and the singlet state oxygen molecules diffuse to the nearby acceptor beads to react with a chemiluminescer in the acceptor bead that further activates fluorophores contained within the same bead. The fluorophores subsequently emit light at 520-620 nm. In the absence of dimerization, the singlet state oxygen molecules produced by the donor bead go undetected without an acceptor bead being held in close proximity. The manufacturer provides detailed protocols and multi-well plate reading devices for using ALPHASCREEN in high throughput formats. A similar homogeneous assay using dimerization of chimeric proteins tagged with an affinity tag and a radioactive tag is the scintillation proximity assay (see Wu S, Liu B.; “Application of scintillation proximity assay in drug discovery” BioDrugs, 2005; 19(6):383-92) supplies, reagents and equipment for which are also commercially available.

Assay results with test agents are preferably compared to positive and negative controls. Thus a plurality any of the cultures described above may be prepared to provide such controls along with test cultures, which are preferably, but not necessarily treated and incubated in parallel. Negative controls typically involve the omission of test agent from the assay. Preferably NS4A-NS4A dimers are not detectible in negative control cells or cultures. Positive controls involve the addition to the assay of an effective amount of a compound known to promote NS4A-NS4A dimerization. Suitable positive control compounds include compounds of acylthiourea and aminothioazole chemotypes (see e.g., Example 7, below) that have been shown to promote homo-dimerization of NS4A, particularly preferred are ACH-806 and the other compounds set forth in Example 7, below. Acylthioureas and aminothioazoles and other compounds of acylthiourea and aminothioazole chemotypes that are not compounds set forth in Example 7, below, and that promote homodimerization of NSA4, are readily identified using the chimeric tagged NSA4 vectors, cells, and assays provided herein. Such compounds are useful in methods of inhibiting HCV replication in vitro or in vivo, which methods involve contacting cells infected with HCV with an amount of such a compound effective to inhibit HCV replication.

### ADDITIONAL SPECIFIC PREFERRED EMBODIMENTS

Different protein tags of the first and the second chimeric tagged HCV NS4A proteins disclosed herein are preferably independently selected from the preferred tags discussed in the SUMMARY OF THE DISCLOSURE above.

With regard to detecting interactions between two proteins that can only be detected when the proteins are joined together by covalent or non-covalent bonds or other non-transient protein-protein connections, and with particular regard to the second group of detectible interactions described in the SUMMARY OF THE DISCLOSURE above as “interaction between protein tags each comprising one of two protein fragments that, when the fragments are joined together in a single linked molecular assembly, can interact in a complementary fashion to provide a detectible function;” a preferred pair of such complementary protein tags are the C-terminal and N-terminal subdomains of ubiquitin used in the split ubiquitn system (Johnson N and Varshaysky A “Split ubiquitin as a sensor of protein interactions in vitro” Proc Natl Acad Sci USA 91 pp 10340-44, 1994). In this system, the two ubiquitin subdomains functionally complement each other only when held in proximity by protein-protein interactions between their two fusion partners, and the complementation results in the auto-proteolytic cleavage of the reconstituted ubiquitin resulting in separation of a reporter moiety linked to the C-terminal ubiquitin subdomain from the subdomain, As applied to first and second chimeric NS4A proteins provided herein, the C-terminal and N-terminal subdomains of ubiquitin serve as the first and second protein tags. With regard to reporter proteins that are linked to the C-terminal ubiquitin subdomain in the split ubiquitin system, a preferred reporter protein is one selected from the fluorescent proteins and fluor-conjugated proteins discussed herein as tags for chimeric NS4A proteins. For example, a preferred reporter protein moiety of a split ubiquitin construct is a tetracysteine tag that is conjugated to biarsenical Fluor (fluorophore). Such fluorescent split ubiquitin reporters can be readily detected in situ as being bound to or cleaved from split ubiquitin by measuring changes in fluorescence anisotropy of the fluor. See, e.g., Blommel P G and Fox B G. “Fluorescence anisotropy assay for proteolysis of specifically labeled fusion proteins” Anal Biochem. 2005 Jan. 1; 336(1):75-86.

In certain embodiments, the protein tag of the first chimeric tagged HCV NS4A protein is a prokaryotic hydrolase variant that is capable of forming a covalent link with a ligand that is a compound of the formula

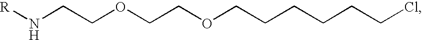

wherein R is selected from a non-fluorescent tagging moiety, such as, FLAG, V5, c-myc, Tab2, HA epitope, biotin, chitin binding protein, maltose binding protein, streptavidin-binding peptide, polystyrene-binding peptide, poly(His) peptide glutathione-s-transferase, or biotin ligase, or a fluorescent tagging moiety. Preferably R is a fluor. A preferred prokaryotic hydrolase variant tag is the PROMEGA HALO tag (PROMEGA, Madison, Wis.) which is conveniently used with its commercially available vectors and ligands (at http://www.promega.com/halotag/ and http://www.promega.com/catalog/catalogproducts.aspx?categoryname=productleaf_{—}1632 as of the international filing date hereof).

In other aspects, a cell may be prepared with a single chimeric tagged HCV NS4A protein comprising the HALO TAG tag, which cell is contacted with a mixture of two HALO ligands having different R groups, often with different functionalities, such as a fluorescent moiety R group and an affinity moiety R group or alternately the two R groups each comprises a different fluor, preferably the two different fluors constituting a FRET pair. Thus, in certain embodiments of these aspects, there is provided a cell that intracellularly expresses a chimeric tagged HCV NS4A protein comprising a HALO TAG protein tag. In this cell, a first fraction of the tagged HCV NS4A protein is covalently bound to a compound of the formula

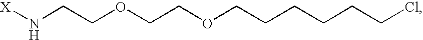

where X is a first fluor, yielding a first fluor-conjugated tagged chimeric HCV NS4A protein, and the first fraction of the tagged chimeric HCV NSA is at a first detectible concentration within the cell, and where a second fraction of the chimeric tagged HCV NS4A protein is covalently bound to a compound of the formula

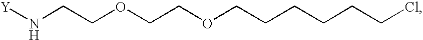

where Y is a second fluor that is not the same as X, yielding a second fluor-conjugated tagged chimeric HCV NS4A protein, and Y is capable of detectible frequency resonance energy transfer to X when chimeric tagged HCV NS4A proteins of the first fraction are dimerized with chimeric tagged HCV NS4A proteins of the second fraction, where the second fraction is at a second detectible concentration within the cell, and where the second concentration is of the same order of magnitude as the first concentration, and is preferably about equimolar to the first concentration.

Alternately, in an analogous fashion to a HALO TAG tag embodiment discussed above, the protein tag of a single chimeric tagged HCV NS4A protein is another tag that can be conveniently bound to ligand by incubation in contact with the ligand. In a preferred embodiment, cells expressing the chimeric tagged protein are incubated with a mixture of a pair two complementary ligands, resulting in a mixed population of tags, one portion of which is conjugated with one of the two complementary ligands, and another portion with another of the pair of complementary ligands. In one embodiment the tag is a tertracysteine tag—a cysteine-cysteine-Xaa-Xaa-cysteine-cysteine (SEQ ID NO:5) tag (where Xaa is an amino acid other than cysteine). A preferred tetracysteine tag is cysteine-cysteine-proline-glycine-cysteine-cysteine (SEQ ID NO:6). Such tags are conveniently conjugated to useful biarsenical tag ligands simply by contacting the tags with the ligands. Preferred biarsenical ligands are fluors, preferably complementary FRET pairs of fluors, which facilitate homogeneous assays that detect dimerization when a FRET signal is obtained upon illumination with light of an appropriate wavelength and intensity. A preferred FRET pair of tetracysteine ligands is F2FlAsH and F4FlAsH. See, e.g., Spagnuolo C C et al., “Improved photostable FRET-competent biarsenical-tetracysteine probes based on fluorinated fluoresceins” J Am Chem Soc. 2006; 128(37):12040-1, also see WO/2007/144077.

A GFP variant, thermotolerant GFP (ttGFP) has two excitation peaks with respective maxima at 395 nm and 475 nm. Illumination of the protein at wavelengths within either peak results in the emission of green light (508 nm) characteristic of GFP. The ratio of the two excitation maxima (the excitation ratio) is the same for any monomeric ttGFP fusion protein when measured in the same buffer and at constant temperature. When ttGFP is fused to a protein that is capable of self-association, the excitation ratio in the homo-dimeric state differs from that of the monomeric state in a characteristic way. These spectral changes can be used to detect the extent of self-association of ttGFP-tagged proteins in vitro and in vivo. See Protein-Protein Interactions: A Molecular Cloning Manual, Golemis E and Adams P D, Editors, Second Edition, Cold Spring Harbor Laboratory Press 2005, Chapter 11, De Angelis, D A, “Detection of Homotypic Protein Interactions with Green Fluorescent Protein Proximity Imaging (GFP-PRIM)” p 215-226.

Thus, there is also provided herein a method for identifying an agent that promotes the homo-dimerization of HCV NS4A protein comprising contacting each of a plurality of cultures of cells that intracellularly expresses chimeric tagged HCV NS4A protein that is tagged with ttGFP with each of a plurality of test agents under conditions allowing interaction between intracellular proteins and each agent; and determining in each culture if the tagged HCV NS4A protein self-associates in a linked molecular assembly after contacting the cell with the test agent so that, when the tagged HCV NS4A protein is determined to have self-associated in a linked molecular assembly after contacting the cell with the test agent, the test agent is identified as promoting homo-dimerization of HCV NS4A protein. The determining of whether the tagged HCV NS4A protein self-associates is preferably made by comparing ratios of GFP excitation maxima at 395 nm to GFP excitation maxima at 475 nm before and after contact with the test agent, and a reproducible difference in the ratio before and after contact with the test agent determines that the tagged HCV NS4A protein self-associates.

Also provided are methods for characterizing the relative extent to which each of a plurality of compounds promotes the homo-dimerization of HCV NS4A proteins, said methods comprising carrying out the above assays separately with each of the plurality of compounds, wherein, in each assay, the presence in a linked molecular assembly of both the first tagged HCV NS4A protein and the second tagged HCV NS4A protein, or the presence in a linked molecular assembly of both the first fluor-conjugated tagged HCV NS4A protein and the second fluor-conjugated tagged HCV NS4A protein or a shift in ratios of excitation maxima of ttGFP, or a change in the fluorescence anisotropy of a fluorescent or fluor-bound tag, is detected as a signal that is assessed for signal intensity, and compounds yielding more intense signals in the assay are characterized as promoting homo-dimerization of HCV NS4A proteins to a greater extent than compounds yielding less intense signals in such an assay.

Each of the references disclosed herein and identified with a beginning and ending page number is incorporated herein by reference for the teachings therein in relation to the subject matter set forth in the individual titles thereof. The Examples that follow are presented for illustrative purposes, and are not to be construed as limiting the scope of the invention(s) disclosed herein.

### EXAMPLES

The ability to inhibit viral replication of Hepatitis C can be tested using the HCV replicon expressed in cultured cells in which an HCV replicon construct has been incorporated. The HCV replicon system was described by Bartenschlager, et. al (Science, 285, pp. 110-113, 1999). The replicon system is predictive of in vivo activity against HCV; compounds that are active in humans uniformly evidence activity in the replicon assay.

In this assay HCV replicon containing cells are treated with different concentrations of the test compound to ascertain the ability of the test compound to suppress replication of the HCV replicon. As a positive control, HCV replicon-containing cells are treated with different concentrations of interferon alpha, a known inhibitor of HCV replication. The replicon assay system includes Neomycin Phosphotransferase (NPT) as a component of the replicon itself as a marker allowing convenient detection of the transcription of replicon gene products in the host cell. Cells in which the HCV replicon is actively replicating have high levels of NPT; the level of NPT is proportional to HCV replication. Cells in which the HCV replicon is not replicating have low levels of NPT and thus do not survive when treated with Neomycin. The NPT level of each sample may be measured using a captured ELISA.

A protocol for testing compounds for the ability to inhibit viral replication of the Hepatitis C replicon cultured cells in which the replicon construct has been incorporated, follows.

### 1A. HCV Replicon and Replicon Expression

The HCV replicon sequence has been deposited in GenBank (Accession no. AJ242652).

The replicon is transfected into Huh-7 cells using standard methods such as electroporation.

### 1B. Cell Maintenance

The equipment and materials include, but are not limited to, Huh-7 HCV replicon-containing cells, maintenance media (DMEM (Dulbecco's modified Eagle media) supplemented with 10% FBS, L-glutamine, non-essential amino acids, penicillin (100 units/ml), streptomycin (100 micrograms/ml), and 500 micrograms/ml of Geneticin G418), screening media (DMEM supplemented with 10% FBS, L-glutamine, and non-essential amino acid, penicillin (100 units/ml) and streptomycin (100 micrograms/ml)), 96 well tissue culture plates (flat bottom), 96 well plates (U bottom for drug dilution), Interferon alpha for positive control, fixation reagent (such as methanol:acetone), primary antibody (rabbit anti-NPTII), secondary antibody: Eu-N1 l, and enhancement solution.

HCV replicon-containing cells support high levels of viral RNA replicon replication when their density is suitable. Over-confluency will cause decreased viral RNA replication. Therefore, cells must be kept growing in log phase in the presence of 500 micrograms/ml of G418. Generally, cells should be passed twice a week at 1: 4-6 dilution. Cell maintenance is conducted as follows:

HCV replicon-containing cells are examined under a microscope to ensure that cells growing well. Cells are rinsed once with PBS and 2 ml trypsin is added. The cell/trypsin mixture is incubated at 37° C. in a CO₂ incubator for 3-5 minutes. After incubation 10 ml of complete media is added to stop the trypsinization reaction. Cells are blown gently, put into a 15 ml tube, and spun at 1200 rpm for 4 minutes. The trypsin/medium solution is removed. Medium (5 ml) is added and the cells are mixed carefully. The cells are counted.

The cells are then seeded onto 96-well plates at a density of 6000-7500 cells/100 microliters/well (6-7.5×105 cells/10 ml/plate). The plates are then incubated at 37° C. in a 5% CO₂ incubator.

Cells are examined under a microscope approximated 24 hours after seeding and prior to adding drugs. If counting and dilution were performed correctly, cells are 60-70% confluent and nearly all cells should attach and spread evenly in the well.

1C. Treatment of HCV-Replicon Containing Cells with Test Compound

HCV replicon-containing cells are rinsed with once PBS once; 2 mls of trypsin is added. Cells are incubated at 37° C. in a 5% CO₂ incubator for 3-5 minutes. 10 mls of complete medium is added to stop the reaction. Cells are blown gently, put into a 15 ml tube, and spun at 1200 rpm for four minutes. The trypsin/medium solution is removed and 5 mls of medium (500 ml DMEM (high glucose)) from BRL catalog #12430-054; 50 mls 10% FBS, 5% Geneticin G418 (50 mg/ml, BRL 10131-035), 5 ml MEM non-essential amino acid (100×BRL #11140-050) and 5 ml pen-strep (BRL #15140-148) is added. The cells and media are mixed carefully

Cells are plated with screening medium (500 ml DMEM (BRL #21063-029), 50 ml FBS (BRL #10082-147) and 5 ml MEM non-essential amino acid (BRL #11140-050) at 6000-7500 cells/100 μl/well of 96 well plate (6-7.5×105 cells/10 ml/plate). Plates are placed into 37° C. 5% CO₂ incubator overnight.

### 1D. Assay

The following morning, drugs (test compounds or interferon alpha) are diluted in 96 well U bottom plates with media or DMSO/media, depending on the final concentration chosen for screening. Generally for 6 concentrations of each test compounds ranging from 10 micromolar to 0.03 micromolar are applied. 100 μl of the test compound dilution is placed in wells of the 96 well plate containing the HCV replicon cells. Media without drug is added to some wells as a negative controls. DMSO is known to affect cell growth. Therefore, if drugs diluted in DMSO are used, all wells, including negative control (media only) and positive control (interferon alpha) wells, must contain the same concentration of DMSO, for single dose screening. The plates are incubated at 37° C. in a humidified 5% CO₂ environment for three days.

On day four, the NTPII assay is quantitated. The medium is poured from the plates and the plates are washed once in 200 μl of PBS. The PBS is then decanted and the plates tapped in a paper towel to remove any remaining PBS. Cells are fixed in situ with 100 μl/well of pre-cooled (−20° C.) methanol:acetone (1:1) and the plates are placed at −20° C. for 30 minutes.

The fixing solution is poured from the plates and the plates allowed to air-dry completely (approximately one hour). The appearance of the dried cell layer is recorded and the density of the cells in the toxic wells is scored with the naked eye. Alternatively cell viability may be assessed using the MTS assay described below.

The wells are blocked with 200 μl of blocking solution (10% FBS; 3% NGS in PBS) for 30 minutes at room temperature. The blocking solution is removed and 100 μl of rabbit anti-NPTII diluted 1:1000 in blocking solution is added to each well. The plates are then incubated 45-60 minutes at room temperature. After incubation, wells are washed six times with PBS-0.05% Tween-20 solution. 100 μl of 1:15,000 diluted Europium (EU)-conjugated goat anti-rabbit in blocking buffer is added to each well and incubated at room temperature for 30-45 minutes. The plates are washed again and 100 μl of enhancement solution (Perkin Elmer #4001-0010) is added to each well. Each plate is shaken (approx. 30 rpm) in a plate shaker for three minutes. 95 μl is transferred from each well to a black plate; the EU signal is quantitated in a Perkin-Elmer VICTOR plate reader (EU-Lance).

### Example 2

### Cytotoxicity Assays

To insure that the decrease in replicon replication detected in the assay of Example 1 is due to compound activity against the HCV replicon rather than nonspecific toxicity a Cellular Protein Albumin Assay or a Cell Proliferation Assay is used to quantitate compound cytotoxicity.

Cellular Protein Albumin Assay: Cellular protein albumin measurements provide a marker of cytotoxicity. The protein levels obtained from cellular albumin assays may also be used to provide a normalization reference for antiviral activity of compounds. In this assay, HCV replicon-containing cells are treated for three days with different concentrations of helioxanthin; a compound that is known to be cytotoxic at high concentrations. The cells are lysed and the cell lysate used to bind plate-bound goat anti-albumin antibody at room temperature (25° C. to 28° C.) for 3 hours. The plate is then washed 6 times with 1×PBS. After washing away the unbound proteins, mouse monoclonal anti-human serum albumin is applied to bind the albumin on the plate. The complex is then detected using phosphatase-labeled anti-mouse IgG as a second antibody.

Cell Proliferation Assay: Cell viability may also be determined by CELLTITER 96 AQUEOUS ONE Solution Cell Proliferation Assay (Promega, Madison Wis.), a colorimetric assay for determining the number of viable cells in a sample. In this method, before fixing the cells, 10-20 μl MTS reagent is added to each well according to manufacturer's instructions, plates are incubated at 37° C. and read at OD 490 nm. During the incubation period living cells covert the MTS reagent to a formazan product which absorbs at 490 nm. Thus the 490 nm absorbance is directly proportional to the number of living cells in culture.

A direct comparison of the Cellular Album and MTS methods for determining cytotoxicity may be obtained as follows: Cells are treated with different concentrations of test compound or Helioxanthin for a three day-period Prior to lysis for detection od album as described above, the MTS reagent is added according to manufacturer's instruction to each well and incubate at 37° C. and read at OD 490 nm. The cellular album assay is then performed as described above.

### Example 3

### Discovery of p14

In this experiment, Huh-7 cells expressing the HCV replicon are treated with the ACH-806 (EC₅₀ 14 nM) for 8 hours. The viral proteins are then immunoprecipitated from cell lysates with anti-NS4A antibodies. Immunoblotting is performed with anti-NS3 or anti-NS4A antibodies following denaturing gel electrophoresis of the immunoprecipitates.

Interestingly, a 14 KDa protein band (p14) is detected in cells treated with ACH-806. A very long exposure shows a small amount of p14 in both the untreated cells and the NS5B inhibitor treated cells; however, the p14 band is substantially more intense in immunoprecipitates from cells treated with ACH-806. An underexposure of the immunoblot shows a decrease in NS4A upon treatment. The large enhancement of the p14 band in the presence of ACH-806 suggests that this protein product may be related to replicase complex inhibition in the presence of the anti-viral compounds such as ACH-806.

The dose-dependence of the p14 band intensity for ACH-806 is assayed to determine if the intensity of the p14 band is directly related to the amount of test compound added. This experiment is performed in the same manner as the previous experiment (i.e., immunoprecipitation with an anti-NS4A antibody followed by immunoblotting with anti-NS4A or anti-NS3) except the ACH-806 concentration is varied as follows: 10 μM, 2 μM, 0.4 μM and 0.08 μM. It was observed that p14 accumulates dose-dependently upon treatment with ACH-806 and the level of NS3 is reduced dose-dependently.

### Example 4

### Preparation of Tagged Chimeric NS4A Vectors

Insert NheI-NS4A-Cflag-EcoRI, coding for a C-terminal V5 epitope tag and having the 5′-3′ sequence: CCCGGCTAGCATGCCCGGCTAGCATGagcacctgggtgctggtaggcggagtcctagcaptctggccgcgta ttgcctgacaacaggcagcgtggtcattgtgggcaggatcatcttgtccggaaagccggccatcattcccgacagggaagtcctttaccg ggagttcgatgagatggaagagtgcGACTACAAAGACGATGACGACAAGTAGGAATTCCGG—SEQ ID NO:7, was ligated into NheI-EcoRI digested expression vector pCl-neo (PROMEGA, Madison, Wis.) to yield vector pCl-neo-NS4A-cFlag.

Insert NheI-NS4A-V5-EcoRI, coding for a C-terminal V5 epitope tag and having the 5′-3′ sequence: CCCGGCTAGCATGagcacctgggtgctggtaggcggagtcctagcagactggccgcgtattgcctgacaacaggcagcgt ggtcattgtgggcaggatcatcttgtccggaaagccggccatcattcccgacagggaagtcctttaccgggagttcgatgagatggaaga gtgcGGTAAGCCTATCCCTAACCCTCTCCTCGGTCTCGATTCTACGTAGGAATTCCGG—SEQ ID NO:8, was ligated into NheI-EcoRI digested expression vector pCl-neo to yield vector pCl-neo-NS4A-cV5.

### Example 5

### Transfection of Huh-7 Cells with Tagged Chimeric NS4A Vectors

4-5×10⁵ Huh-7 cells are transfected in about 3 mL of medium in each well of a 6-well plate. 12 ul of the lipofection reagent FUGENE HD (Roche Diagnostic Products), 2 ug of pCl-neo-NS4A-cV5 and 0.2 ug of pCl-neo-NS4A-cFlag are added and incubated in accordance with the FUGENE HD manufacturer's directions.

When the vector is a viral vector it may be introduced into the cell by transduction. One example of a viral vector is the Lentivirus transduction system, in which virus is produced harboring the gene of interest (in this case tagged-NS4A). The cell line of interest (huh-7, CHO, etc.) is transduced by infection with this virus. This is produces a stably transfected cell line after serial passaging removes the unwanted viral remnants.

### Example 6

### FLISA and ELISA

Cells co-transfected with pCl-neo-NS4A-cV5 and pCl-neo-NS4A-cFlag as described in the previous Example are grown to about 50-75% confluency in a culture and are then incubated in culture medium comprising from about 1 to about 10 (preferably about 5) micromolar test agent for about 20 hours. Culture medium is then removed and the cultured cells are then lysed with a suitable amount (depending on the quantity of cells in the culture) of detergent lysis buffer.

An aliquot of the lysate is added to a well of a multiwell polystyrene plate that has been pre-coated with anti-V5 antibody. The aliquot is of a volume at least adequate to completely cover the bottom of the well.

After incubation for 0.1-5 hours, the lysate is removed, the plate washed with buffer, and a suitable buffer containing an appropriate dilution (per the manufacturer's instructions) of fluor- (rendering this assay a FLISA) or enzyme- (rendering this assay a ELISA) congugated anti-FLAG antibody is added. Following incubation according to the antibody vendor's instructions, the wells are washed. If a FLISA, fluorescence in the wells is then read with a suitable instrument. If an ELISA, a suitable reaction mixture for detection of activity of the enzyme linked to the antibody is added and enzymatic activity is assessed by measuring the amount of a product of enzymatic action on the substrate in the reaction mixture.

### Example 7

### Tested Compounds

The following compounds, the first of which is ACH-806, have been tested in an NS4A dimeriazation assay using the transfected cells of Example 5 and identified as agents promoting homo-dimerization of NS4A. These compounds have also been tested in the replicon assay of Example 1 and are found to inhibit replicon replication, while not exhibiting substantial cytotoxicity in an assay of Example 2.

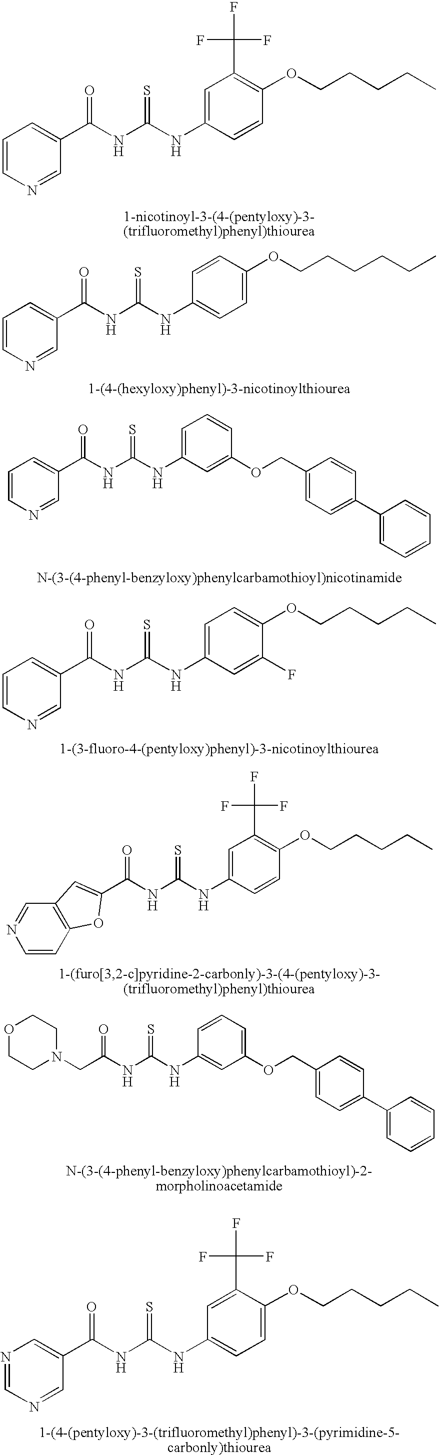

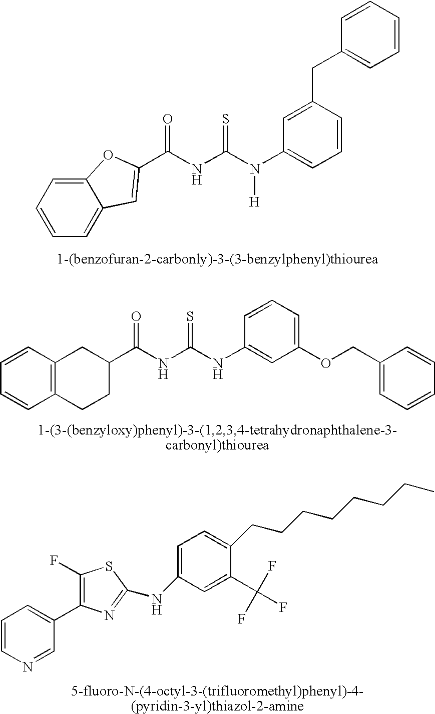

### Example 8

### Characterization of Replicon Variants Resistant to ACH-806

### 8A. Genotypic Analysis of Resistant Replicon Variants.

To a certain if the reduced ACH-806 susceptibility of the resistant replicon cellular lines was due to distinctive replicon mutation(s), we determined the sequences of the entire nonstructural protein-coding regions of replicons extracted from the 3 resistant replicon cellular lines (#22, #24 and #28 from 1st selection) and aligned them with the sequences of the parental replicon. Besides some non-conserved mutations randomly scattering throughout the replicons, we found two sense mutations that were conserved in all three replicons isolated from resistant replicon cellular lines. One mutation resulted in the replacement of cysteine 16 of NS3 (residue 1042 of the polyprotein) with serine (C16S) (Table 1), and the other the replacement of isoleucine 585 of NS5B (residue 3004 of the polyprotein) with threonine (I585T). Since the C16S mutation in NS3 was the mutation conferring resistance to ACH-806 (see below), the subsequent genetic analysis focused on the NS3 region. After sequencing the NS3 region of replicons extracted from the remaining 5 resistant replicon cellular lines, we found that two of them (#23 and #25 from 1st selection) carried the C16S mutation, however, the other 3 (#6, #8 and #9 from 2nd selection) carried a different mutation that replaced alanine 39 of NS3 (residue 1065 of the polyprotein) with valine (A39V) (Table 1). The same A39V mutation was previously detected in resistant replicon variants selected with several ACH-806 analogs and was identified to be responsible for the reduced susceptibility of the resistant replicon cellular clones to those analogs (data not shown).

### 8B. Determination of Mutations Responsible for the Resistant Phenotype.

To investigate the genetic basis of resistance to ACH-806 in the resistant replicon cellular lines, specifically the role of C16S and A39V in NS3, and 1585T 1 in NS5B, we introduced these mutations separately into a nonselectable replicon containing a luciferase reporter gene. Following the transfection of the parental replicon RNA molecule and the C16S, A39V and I585T variant replicon RNA molecules into Huh-7 cells, the replication capacity of all replicons was determined as described in Method and Materials. Replicon RNA carrying the A39V mutation replicated as efficiently as its parent, whereas the C16S and I585T mutations caused a slight decrease in the replication capacity (about 75% that of the parental replicon). The susceptibility of these four replicons to ACH-806 as well as to other classes of inhibitors was compared side by side, and the results are summarized in Table 2. The I585T mutation did not significantly affect the potency of any compound. The C16S and A39V mutations, on the other hand, increased EC50 values of ACH-806, 12 and 14 fold respectively. In contrast, neither of the mutations significantly affected the potency of other control inhibitors. In addition, a replicon variant carrying both the C16S and A39V mutations was also made 15 but failed to replicate efficiently in Huh-7 cells.

To further confirm the role of C16S and A39V in conferring resistance to ACH-806, we introduced both mutations, separately, into a selectable replicon which carries the neo gene and the adaptive mutations that differ from the nonselectable replicon. Following transfection of the parental replicon RNA, the C16S replicon RNA and A39V replicon RNA into Huh-7 cells, the susceptibility of these three replicons to ACH-806, as evaluated by the number of colonies formed under the treatment of both G418 and ACH-806, was compared side by side. Again, an approximately 10-fold reduction in the susceptibility of C16S or A39V replicon variant to ACH-806 was observed.

A third approach, fragment cloning, was used to reinforce the role of the C16S mutation and to exclude the role of other nonconsensus mutations in conferring resistant to ACH-806. A ˜3 kb fragment covering the coding region of NS3, NS4A, and part of NS4B was amplified by RT-PCR from total RNA isolated from ACH-806 resistant clone #28. The PCR product was cloned into the nonselectable replicon containing a luciferase reporter gene. Replicon RNA molecules were made from eight individual clones and were transfected into Huh-7 cells to determine the replication capacity by luciferase activity. From the eight clones, two were able to replicate efficiently and sequencing of the entire cloned fragment in these two clones confirmed the existence of C16S mutation in addition to other nonconsensus mutations. The serine at the amino acid residue of NS3 in one of these two clones was then converted back to cysteine using site-directed mutagenesis, leaving other nonconsensus mutations unchanged. The replication capacity of this resulting replicon [Luc/N3-4AR(C16)] was 95% that of its parent [Luc/N3-4AR(S16)] determined as previously described. After transfection into Huh-7 cells, the susceptibility of both replicons was compared. The reversion of serine back to cysteine at amino acid residue 16 of NS3 increased the susceptibility of the replicon to ACH-806 about 10 fold and maintained the susceptibility to other classes of HCV inhibitors (Table 3). Similar results were also obtained with a fragment containing NS3, 4A and part of 4B derived from a resistant cell line carrying A39V mutation (data not shown). In summary, all three approaches yielded a 10-15 fold decrease in the susceptibility to ACH-806 in the replicon variants carrying 1 either the C16S or A39V mutation, a value similar to what was observed for the resistant cell clones emerging under the selection of ACH-806 (Table 1). Hence, we conclude that either the C16S or A39V mutation in HCV replicon is sufficient to confer the replicon resistance to ACH-806.

### 8C. No Cross Resistance Between ACH-806 and Other Classes of HCV Inhibitors.

In the study described above, we showed that there was no significant difference in the susceptibility between ACH-806 resistant replicon variants and their parental replicon to the other classes of HCV inhibitors (Table 2-3). In addition, the susceptibility of ACH-806 resistant replicon cellular lines to IFN and ribavirin was also similar to the parent cell line (data not shown). The lack of cross resistance between ACH-806 and other classes of HCV inhibitors was further confirmed by evaluation of the susceptibility to ACH-806 of replicon cellular lines resistant to other classes of HCV inhibitors. The replicon cellular lines resistant to each of HCV inhibitors were obtained by selection in Huh-9-13 cell line resistant replicon cellular lines were highly resistant to their inducing agent (fold change in EC50>20) and carried the signature mutations reported previously by others (23,31,36,39,49,52) (Table 4). No significant difference was observed when the susceptibility to ACH-806 of these resistant replicon cellular lines and the parental replicon cellular line was compared side by side (Table 4). Hence, we conclude that ACH-806 is not cross resistant with the other classes of inhibitors that we have tested, including nucleoside and nonnucleoside NS5B polymerase inhibitors, and NS3 protease inhibitors.

TABLE 1
Phenotype and genotype of ACH-806 resistant replicon cellular lines

| | Fold change^{a} in EC₅₀ | | | | Codon at | Codon at |
|---|---|---|---|---|---|---|
| | ACH- | | | | residue 16 | residue of |
| Cell lines | 806 | NNI-1 | PI-1 | NI-1 | of NS3 | 39 of NS3 |
| Parental | 1.0 | 1.0 | 1.0 | 1.0 | UGC(Cys) | GCA(Ala) |
| 806^{R} | 13.9 | 2.2 | 2.2 | 2.2 | AGC(Ser) | GCA(Ala) |
| #22(Selection | | | | | | |
| 1) | | | | | | |
| 806^{R} | 18.6 | .3 | 2.5 | ND^{c} | AGC(Ser) | GCA(Ala) |
| #23(Selection | | | | | | |
| 1) | | | | | | |
| 806^{R} | 11.2 | 1.3 | 1.2 | 2.0 | AGC(Ser) | GCA(Ala) |
| #24(Selection | | | | | | |
| 1) | | | | | | |
| 806^{R} | 14.9 | .9 | 1.1 | ND^{c} | AGC(Ser) | GCA(Ala) |
| #25(Selection | | | | | | |
| 1) | | | | | | |
| 806^{R} | 21.3 | .8 | .8 | 3.0 | AGC(Ser) | GCA(Ala) |
| #28(Selection | | | | | | |
| 1) | | | | | | |
| 806^{R} | 18.6 | 2.6 | 4.2 | 2.0 | UGC(Cys) | GTA(Val) |
| #6(Selection | | | | | | |
| 2) | | | | | | |
| 806^{R} | 9.5 | 1.2 | 1.1 | 1.9 | UGC(Cys | GTA(Val) |
| #8(Selection | | | | | | |
| 2) | | | | | | |
| 806^{R} | 11.5 | 3.0 | 2.7 | 2.4 | UGC(Cys) | GTA(Val) |
| #9(Selection | | | | | | |
| 2) | | | | | | |
| Average fold | 14.9 | 1.3 | 1.7 | 2.7 | — | — |
| change | | | | | | |

^{a}Fold change in EC₅₀ is calculated by comparison of that seen with the parental replicon cellular line, Huh-9-13.
^{b}EC₅₀ ± SD in μM from 3 independent experiments against Huh-9-13 are: 0.04 ± 0.02 (ACH806), 0.78 ± 0.2.
^{c}ND, not determined

TABLE 2
Susceptibility of the parental and mutant replicon variants to ACH-806 and control
inhibitors^{a}

| | Potency (μM) | | | | Fold change in potency^{b} | | | |
|---|---|---|---|---|---|---|---|---|
| | | | Luc/NS3- | | Luc/ | | | |
| | Luc/Parent | | A39V | | NS3-C16S | | | |
| | replicon | | replicon | | replicon | | Luc/NS5B-I585T | |
| Compound | EC₅₀ | EC₉₀ | EC₅₀ | EC₉₀ | EC₅₀ | EC₉₀ | EC₅₀ | EC₉₀ |
| ACH-806 | 0.01 ± 0.01 | 0.08 ± 0.01 | 14.0 | 13.7 | 11.1 ± 1.0 | 11.3 ± 0.9 | 0.7 ± 0.1 | 1.1 ± 0.1 |
| NNI-1 | 1.76 ± 0.56 | 7.50 ± 1.00 | 1.0 | 1.1 | 1.1 ± 0.2 | 1.0 ± 0.1 | 1.3 ± 0.4 | 1.1 ± 0.2 |
| PI-1 | 0.90 ± 0.88 | 4.77 ± 2.10 | 1.3 | 0.7 | 0.8 ± 0.4 | 1.1 ± 0.6 | 0.8 ± 0.4 | 1.0 ± 0.0 |
| NI-1 | 0.14 ± 0.06 | 1.25 ± 0.44 | 0.9 | 1.2 | 1.2 ± 0.6 | 0.9 ± 0.2 | 1.4 ± 0.8 | 1.0 ± 0.0 |

^{a}Data shown are averages ± standard deviations of results from two independent experiments except ones for the replicon Luc/NS3-A39V where only one experiment was conducted.
^{b}Fold change in EC₅₀ and EC₉₀ is calculated by comparison of that seen with Luc/Parent replicon.

TABLE 3
Susceptibility of the mutant replicon variants with and without C16S mutation to
ACH-806 and control inhibitors^{a}

| | Luc/NS3-4A^{R(S16)} | | Luc/NS3-4A^{R(C16)} | | | |
|---|---|---|---|---|---|---|
| | replicon | | replicon | | FC^{b}in | |
| Compound | EC₅₀ (μM) | EC₉₀ (μM) | EC₅₀ (μM) | EC₉₀ (μM) | EC₅₀ | EC₉₀ |
| ACH-806 | 0.27 ± 0.04 | 2.35 ± 0.24 | 0.02 ± 0.00 | 0.24 ± 0.01 | 13.5 | 9.8 |
| NNI-1 | 0.44 ± 0.13 | 2.84 ± 0.06 | 0.63 ± 0.03 | 3.04 ± 0.05 | 0.7 | 0.9 |
| PI-1 | 0.38 ± 0.10 | 1.88 ± 0.08 | 0.64 ± 0.05 | 2.52 ± 0.03 | 0.6 | 0.7 |
| NI-1 | 0.19 ± 0.00 | 0.86 ± 0.01 | 0.24 ± 0.01 | 0.90 ± 0.01 | 0.8 | 1.0 |

^{a}Data shown are averages ± standard deviations of results from two independent experiments.
^{b}FC, fold change in EC₅₀ or EC₉₀ over that seen with Luc/NS3-4^{R(C16)} replicon.

TABLE 4
Susceptibility of other resistant cell lines to ACH-806

| Cell | Fold change in EC₅₀^{b} | | | | | |
|---|---|---|---|---|---|---|
| lines (Resistant | ACH- | | BILN | | | |
| mutation) | 806 | VX-950 | 2061 | NM107 | NI-1 | NNI-1 |
| Parental | 1.0 | 1.0 | 1.0 | 1.0 | 1.0 | 1.0 |
| VX-950^{R} | 0.6 | 43.7 | >100 | ND^{d} | 0.6 | ND^{d} |
| (A156T)^{c} | | | | | | |
| BILN 2061^{R} | 1.1 | 2.9 | >100 | ND^{d} | 1.4 | 0.8 |
| (D168V)^{c} | | | | | | |
| NM107^{R} | 0.5 | 0.3 | 1.1 | 30.7 | 11.5 | 2.9 |
| (S282T)^{c} | | | | | | |
| NI-1^{R} (S282T)^{c} | 0.8 | ND | 1.4 | >68.7 | 30.3 | 5.0 |
| NNI-1^{R} (M414T)^{c} | 0.9 | 2.3 | 2.8 | ND^{d} | 0.6 | >28.4 |

^{a}Fold change in EC₅₀ is calculated by comparison of that seen with the parent replicon cellular lones, Huh-9-13.
^{b}EC₅₀ ± SD from 2 independent experiments against Huh-9-13 are: 0.04 ± 0.03 μM (ACH-806), 0.61 ± 9.14 μM (VX-950), 1.2 ± 0.61 nM (BILN 2061), 1.16 ± 1.22 μM (NM 107), 0.56 ± 0.17 μM (NI-1), and 1.37 ± 0.56 μM (NNI-1).
^{c}The specific mutations detected in the various inhibitor-resistant replicons are indicated in the parenthesis.
^{d}ND, not determined

1. A cell, wherein the cell intracellularly co-expresses a first chimeric tagged HCV NS4A protein and a second chimeric tagged HCV NS4A protein,
wherein each of the first chimeric tagged HCV NS4A protein and the second chimeric tagged HCV NS4A protein comprises an N-terminal end and a C-terminal end and each further comprises a contiguous amino acid sequence consisting essentially of HCV NS4A protein and a protein tag,
wherein the protein tag of the first chimeric tagged HCV NS4A protein is a different protein tag than the protein tag of the second chimeric tagged HCV NS4A protein.

2. The cell of claim 1 wherein the first chimeric tagged HCV NS4A protein is expressed at a first concentration within the cell and the second chimeric tagged HCV NS4A protein is expressed at a second concentration within the cell, said first concentration being within about an order of magnitude of said second concentration.

3. (canceled)

4. The cell of claim 2 wherein the different protein tags of the first and the second chimeric tagged HCV NS4A proteins are independently selected from an epitope tag, a fluorescence tag, an affinity tag, and an enzymatic tag.

5-10. (canceled)

11. A cell, wherein the cell intracellularly expresses a chimeric tagged HCV NS4A protein comprising a prokaryotic hydrolase variant that is capable of forming a covalent link with a compound of the formula 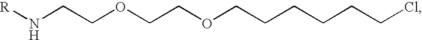
wherein R is selected from Biotin, PEG-Biotin, or a fluor;
wherein a first fraction of the tagged HCV NS4A protein is covalently bound to the compound of the formula 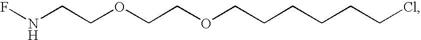
wherein F is a fluor and said first fraction is present in the cell at a first detectible concentration; and
wherein a second fraction of the chimeric tagged HCV NS4A protein is covalently bound to a compound of the formula 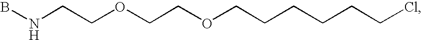
wherein B is biotin or PEG-biotin and said second fraction is at a second detectible concentration within the cell, and
wherein said second concentration is of the same order of magnitude as said first concentration.

12-16. (canceled)

17. The cell of claim 1, wherein the cell is a cultured mammalian cell.

18-19. (canceled)

20. The cell of claim 4, wherein the cell is a Huh-7 cell, the protein tag of the first chimeric tagged HCV NS4A is a V5 tag, and the protein tag of the second chimeric tagged HCV NS4A is a FLAG tag.

21-30. (canceled)

31. An assay method for identifying substances that promote the homo-dimerization of HCV NS4A proteins,
said assay comprising incubating the culture of cells of claim 28 with a compound and detecting the presence or absence of dimers of the chimeric tagged HCV NS4A proteins in the cells of the culture of a lysate thereof,
wherein, detection of the presence of dimers of the chimeric tagged HCV NS4A is achieved by detecting the presence in a linked molecular assembly of both the first tagged HCV NS4A protein and the second tagged HCV NS4A protein, or the presence in a linked molecular assembly of both the first fluor-conjugated tagged HCV NS4A protein and the second fluor-conjugated tagged HCV NS4A protein,
wherein, detection of the presence of dimers of the chimeric tagged HCV NS4A proteins upon incubation of the culture with the compound identifies the compound as promoting the homo-dimerization of HCV NS4A proteins.

32. A method for characterizing the relative extent to which each of a plurality of compounds promotes the homo-dimerization of HCV NS4A proteins,
said method comprising carrying out the assay of claim 31 with each of the plurality of compounds, wherein, in the assay, the presence in a linked molecular assembly of both the first tagged HCV NS4A protein and the second tagged HCV NS4A protein, or the presence in a linked molecular assembly of both the first fluor-conjugated tagged HCV NS4A protein and the second fluor-conjugated tagged HCV NS4A protein is detected as a signal that is assessed for signal intensity, and compounds yielding more intense signals in the assay are characterized as promoting homo-dimerization of HCV NS4A proteins to a greater extent than compounds yielding less intense signals in the assay.

33. (canceled)

34. A method for identifying an agent that promotes the homo-dimerization of HCV NS4A protein, said method comprising: contacting a cell that intracellularly expresses two differently protein-tagged HCV NS4A proteins with a test agent under conditions allowing interaction between intracellular proteins and the agent; and determining whether the two differently tagged HCV NS4A proteins become associated together in a linked molecular assembly after contacting the cell with the test agent, wherein, when the two differently tagged HCV NS4A proteins are determined to have become associated together in a linked molecular assembly after contacting the cell with the test agent, the test agent is identified as promoting homo-dimerization of HCV NS4A protein.

35. The method of claim 34, wherein the cell does not comprise a hepatitis C virus replication complex.

36. (canceled)

37. The method of claim 34, wherein each protein-tagged HCV NS4A protein is expressed from one or more expression vectors, each directing the expression of one or more protein-tagged HCV NS4A proteins, which one or more vectors is the same vector or a different vector for each protein-tagged HCV NS4A protein.

38-41. (canceled)

42. The method of claim 34, wherein the cell is a human hepatocellular carcinoma Huh-7 cell, an African green monkey COS cell, or a Chinese hamster ovary CHO cell.

43. The method of claim 34, wherein the agent is of an acylthiourea chemotype or an aminothioazole chemotype.

44. The method of claim 34, wherein the method comprises further testing the properties of the agent identified as promoting homo-dimerization of HCV NS4A protein, wherein the further testing consists of assessing the degree to which the agent inhibits HCV replication in a replicon-based assay in cultured cells.

45. (canceled)

46. An expression vector wherein the vector is adapted for functions comprising replication in prokaryotic cells and protein expression in eukaryotic cells, said vector expressing, upon introduction into a compatible eukaryotic cell, a first chimeric tagged HCV NS4A protein and a second chimeric tagged HCV NS4A protein,
said vector comprising a first discrete sequence encoding the first chimeric tagged HCV NS4A protein and a second discrete sequence encoding the second chimeric tagged HCV NS4A protein, the first discrete sequence being functionally linked to a first discrete eukaryotic promoter and the second discrete sequence being linked to a second discrete eukaryotic promoter, which second promoter may have a sequence that is the same as or different from the sequence of the first promoter;
wherein each of the first chimeric tagged HCV NS4A protein and the second chimeric tagged HCV NS4A protein comprises an N-terminal end and a C-terminal end and each further comprises a contiguous amino acid sequence consisting essentially of HCV NS4A protein and a protein tag,
wherein the protein tag of the first chimeric tagged HCV NS4A protein is a different protein tag than the protein tag of the second chimeric tagged HCV NS4A protein.

47-48. (canceled)

49. The vector of claim 46 wherein, upon introduction into the compatible eukaryotic cell, the first chimeric tagged HCV NS4A protein is expressed at a first concentration within the cell and the second chimeric tagged HCV NS4A protein is expressed at a second concentration within the cell, said first concentration being within about an order of magnitude of said second concentration.

50. (canceled)

51. The vector of claim 46 wherein the different protein tags of the first and the second chimeric tagged HCV NS4A proteins are independently selected from an epitope tag, a fluorescence tag, an affinity tag, and an enzymatic tag.

52. The vector of claim 46 wherein the different protein tags of the first and the second chimeric tagged HCV NS4A proteins are independently selected from a FLAG, V5, c-myc, Tab2, or HA epitope tag, a GFP, YFP, CFP, or OFP fluorescence tag, a luciferase tag, a chitin binding protein, maltose binding protein, streptavidin-binding peptide, polystyrene-binding peptide, or poly(His) affinity tag, and a glutathione-s-transferase, hydrolase, or biotin ligase enzymatic tag.

53-54. (canceled)

55. A method of using the vector of claim 46, said method comprising introducing the vector into a mammalian cell so that the first and the second chimeric HCV NSA2 proteins are expressed therein.

56. The method of claim 4 wherein the vector is introduced into the cell by transfection or transduction using a viral vector.

57-59. (canceled)


SEQUENCE LISTING (8 sequences)

SEQ ID NO: 1 - DNA, 162 bp, Hepatitis C virus
agcacctgggtgctggtaggcggagtcctagcagctctggccgcgtattgcctgacaacaggcagcgtggtcattgtgggcaggatcatcttgtccggaaagccggccatcattcccgacagggaagtcctttaccgggagttcgatgagatggaagagtgc

SEQ ID NO: 2 - protein, 54 aa, Hepatitis C virus
STWVLVGGVLAALAAYCLTTGSVVIVGRIILSGKPAIIPDREVLYREFDEMEEC

SEQ ID NO: 3 - protein, 8 aa, Artificial Sequence
  FLAG tag
DYKDDDDK

SEQ ID NO: 4 - protein, 14 aa, Artificial Sequence
  V5 tag
GKPIPNPLLGLNST

SEQ ID NO: 5 - protein, 6 aa, Artificial Sequence
  Tetracysteine tag
CCXXCC

SEQ ID NO: 6 - protein, 6 aa, Artificial Sequence
  tetracysteine tag
CCPGCC

SEQ ID NO: 7 - DNA, 224 bp, Artificial Sequence
  DNA coding for V5 epitope tag
cccggctagcatgcccggctagcatgagcacctgggtgctggtaggcggagtcctagcagctctggccgcgtattgcctgacaacaggcagcgtggtcattgtgggcaggatcatcttgtccggaaagccggccatcattcccgacagggaagtcctttaccgggagttcgatgagatggaagagtgcgactacaaagacgatgacgacaagtaggaattccgg

SEQ ID NO: 8 - DNA, 229 bp, Artificial Sequence
  DNA coding for V5 epitope tag
cccggctagcatgagcacctgggtgctggtaggcggagtcctagcagctctggccgcgtattgcctgacaacaggcagcgtggtcattgtgggcaggatcatcttgtccggaaagccggccatcattcccgacagggaagtcctttaccgggagttcgatgagatggaagagtgcggtaagcctatccctaaccctctcctcggtctcgattctacgtaggaattccgg